SEQUENCE LISTING (14 sequences)

SEQ ID NO: 1 - DNA, 35938 bp, Adenovirus type 5
  source 1..35938: mol_type = unassigned DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 2 - DNA, 10 bp, synthetic construct
  misc_feature 1..10: note = Sequence resulting from TAV-255 deletion
  source 1..10: mol_type = other DNA
ggtgttttgg

SEQ ID NO: 3-5 - skipped (3 entries)

SEQ ID NO: 6 - DNA, 46 bp, synthetic construct
  misc_feature 1..46: note = Exempalry modified E1b-19k region
  source 1..46: mol_type = other DNA
atcttggttacatctgacctcgtcgagtcaccaggcgcttttccaa

SEQ ID NO: 7 - DNA, 1185 bp, synthetic construct
  misc_feature 1..1185: note = mTGFB-Trap
  source 1..1185: mol_type = other DNA
atgggtcgggggctgctccggggcctgtggccgctgcatatcgtcctgtggacgcgcatcgccagcacgatcccgccgcacgttcccaagtcggttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgacagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccgcaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 8-9 - skipped (2 entries)

SEQ ID NO: 10 - DNA, 10 bp, synthetic construct
  misc_feature 1..10: note = Sequence resulting from exemplary E1A promoter CAAT box deletion
  source 1..10: mol_type = other DNA
ttccgtggcg

SEQ ID NO: 11 - skipped

SEQ ID NO: 12 - DNA, 10 bp, Adenovirus type 5
  source 1..10: mol_type = unassigned DNA
taataaaaaa

SEQ ID NO: 13 - skipped

SEQ ID NO: 14 - DNA, 11 bp, Adenovirus type 5
  source 1..11: mol_type = unassigned DNA
taaaaaaaaat

(19) United States
(12) Patent Application Publication
(10) Pub. No.: US 2024/0294883 A1
(43) Pub. Date: Sep. 5, 2024

(54) COMPOSITIONS FOR PRODUCING RECOMBINANT VIRUS

(71) Applicant: EpicentRx, Inc., La Jolla, CA (US)

(72) Inventors: Tony R. REID, San Diego, CA (US); Bryan T. ORONSKY, Los Altos Hills, CA (US); Christopher LARSON, San Diego, CA (US)

(73) Assignee: EpicentRx, Inc., La Jolla, CA (US)

(21) Appl. No.: 18/649,467

(22) Filed: Apr. 29, 2024

Related U.S. Application Data
(63) Continuation of application No. 16/604,194, filed on Oct. 10, 2019, now Pat. No. 11,999,973.
(60) Provisional application No. 62/483,837, filed on Apr. 10, 2017.

(51) Int. Cl. C12N 7/00

(52) U.S. Cl. CPC C12N 7/00; C12N 2710/10321; C12N 2710/10322; C12N 2710/10332; C12N 2710/10352

(57) ABSTRACT

The present disclosure relates to compositions and methods for producing a recombinant virus, e.g., a recombinant oncolytic adenovirus, using an A549 host cell.

### CROSS-REFERENCE TO RELATED APPLICATIONS

This application is a continuation of U.S. application Ser. No. 16/604,194, which is a U.S. National Phase patent application under 35 U.S.C. § 371 of International Application No. PCT/US2018/026977, filed internationally on Apr. 10, 2018, which claims the priority benefit of U.S. Provisional Application Ser. No. 62/483,837, filed Apr. 10, 2017, the disclosures of which is are incorporated herein by reference in its their entirety.

### REFERENCE TO AN ELECTRONIC SEQUENCE LISTING

The contents of the electronic sequence listing (203592001201SEQLIST.xml; Size: 47,277 bytes; and Date of Creation: Apr. 23, 2024) is herein incorporated by reference in its entirety.

### TECHNICAL FIELD

The present disclosure relates to compositions and methods for producing a recombinant virus, e.g., a recombinant oncolytic adenovirus.

### BACKGROUND

Despite extensive knowledge of the underlying molecular mechanisms that cause cancer, most advanced cancers remain incurable with current chemotherapy and radiation protocols. Oncolytic viruses have emerged as a platform technology that has the potential to significantly augment current standard treatment for a variety of malignancies (Kumar, S. et al. (2008) CURRENT OPINION IN MOLECULAR THERAPEUTICS 10(4):371-379; Kim, D. (2001) EXPERT OPINION ON BIOLOGICAL THERAPY 1(3):525-538; Kim D. (2000) ONCOGENE 19(56):6660-6669). These viruses have shown promise as oncolytic agents that not only directly destroy malignant cells via an infection-to-reproduction-to-lysis chain reaction but also indirectly induce anti-tumor immunity. These immune stimulatory properties have been augmented with the insertion of therapeutic transgenes that are copied and expressed each time the virus replicates.

Previously developed oncolytic viruses include the oncolytic serotype 5 adenovirus (Ad5) referred to as TAV-255 that is transcriptionally attenuated in normal cells but transcriptionally active in cancer cells (see, PCT Publication No. WO2010/101921). It is believed that the mechanism by which the TAV-255 vector achieves this tumor selectivity is through targeted deletion of three transcriptional factor (TF) binding sites for the transcription factors Pea3 and E2F, proteins that regulate adenovirus expression of E1a, the earliest gene to be transcribed after virus entry into the host cell, through binding to specific DNA sequences.

Despite the efforts to date, there is a need for improved viruses for treating cancers and hyperproliferative disorders in human patients, and improved methods for producing recombinant viruses.

### SUMMARY OF THE INVENTION

The invention is based, in part, upon the discovery that an A549 host cell, e.g., a SF-BMAdR 281 A549 host cell, can be used to produce large quantities of a recombinant virus, e.g., an oncolytic adenovirus. It has surprisingly has been found that certain recombinant viruses, e.g., recombinant oncolytic adenoviruses, grow to higher densities in a replication permissive environment in serum-free and suspension-adapted A549 cells than in HEK293 cells, which are widely used for viral vector production.

Accordingly, in one aspect, the invention provides a method for producing a recombinant virus comprising: (a) infecting an A549 host cell with a recombinant virus to produce an infected A549 host cell; and (b) suspension culturing the infected A549 host cell in a serum-free medium, under conditions (e.g., in a replication permissive environment) to permit replication of the recombinant virus, thereby to produce the recombinant virus. In certain embodiments, the A549 host cell is a SF-BMAdR 281 A549 host cell. In certain embodiments, the infected A549 host cell is cultured for at least 3 days.

The method may further comprise, after step (b), the step of purifying the recombinant virus. The step of purifying the recombinant virus may comprise one or more of lysing the infected A549 host cell, nuclease treatment, and ion exchange chromatography, e.g., anion exchange chromatography. In certain embodiments, the step of purifying the recombinant virus comprises: (i) lysing the infected A549 host cell to produce a cell lysate; (ii) treating the cell lysate with nuclease to produce a treated cell lysate; and (iii) purifying the recombinant virus from the treated cell lysate by ion exchange chromatography, e.g., anion exchange chromatography.

The method may result in a greater yield of recombinant virus than a comparable method for producing a recombinant virus. For example, in certain embodiments, the method results in at least 5×, 10×, or 20× more recombinant virus compared to a similar method (e.g., an otherwise identical method) that comprises, in step (a), infecting a HEK293 host cell with a recombinant virus to produce an infected HEK293 host cell, and, in step (b), suspension culturing the infected HEK293 host cell in a serum-free medium, under conditions (e.g., in a replication permissive environment) to permit replication of the recombinant virus. In certain embodiments, the method results in at least 5×, 10×, or 20× more recombinant virus compared to a similar method (e.g., an otherwise identical method) that comprises, in step (b), adherent culturing the infected A549 host cell in a serum-free medium, under conditions (e.g., in a replication permissive environment) to permit replication of the recombinant virus. In certain embodiments, the method results in at least 5×, 10×, or 20× more recombinant virus compared to a similar method (e.g., an otherwise identical method) that comprises, in step (b), suspension culturing the infected A549 host cell in a serum-containing medium, under conditions (e.g., a replication permissive environment) to permit replication of the recombinant virus.

In certain embodiments, the recombinant virus is an adenovirus, e.g., a type 5 adenovirus, or an adeno-associated virus. In certain embodiments, the recombinant virus is a recombinant oncolytic virus. In certain embodiments, the recombinant virus is a recombinant oncolytic adenovirus.

In another aspect, the invention provides a method for producing a recombinant oncolytic adenovirus comprising: (a) infecting an A549 host cell with a recombinant oncolytic adenovirus to produce an infected A549 host cell; and (b) suspension culturing the infected A549 host cell in a serum-free medium, under conditions (e.g., in a replication permissive environment) to permit replication of the recombinant oncolytic adenovirus, thereby to produce the recombinant oncolytic adenovirus. In certain embodiments, the A549 host cell is a SF-BMAdR 281 A549 host cell. In certain embodiments, the infected A549 host cell is cultured for at least 3 days.

The method may further comprise, after step (b), the step of purifying the recombinant oncolytic adenovirus. The step of purifying the recombinant oncolytic adenovirus may comprise one or more of lysing the infected A549 host cell, nuclease treatment, and ion exchange chromatography, e.g., anion exchange chromatography. In certain embodiments, the step of purifying the recombinant oncolytic adenovirus comprises: (i) lysing the infected A549 host cell to produce a cell lysate; (ii) treating the cell lysate with nuclease to produce a treated cell lysate; and (iii) purifying the recombinant virus from the treated cell lysate by ion exchange chromatography, e.g., anion exchange chromatography.

The method may result in a greater yield of recombinant oncolytic adenovirus than a comparable method for producing a recombinant oncolytic adenovirus. For example, in certain embodiments, the method results in at least 5×, 10×, or 20× more recombinant oncolytic adenovirus compared to a similar method (e.g., an otherwise identical method) that comprises, in step (a), infecting a HEK293 host cell with a recombinant oncolytic adenovirus to produce an infected HEK293 host cell, and, in step (b), suspension culturing the infected HEK293 host cell in a serum-free medium, under conditions (e.g., in a replication permissive environment) to permit replication of the recombinant oncolytic adenovirus. In certain embodiments, the method results in at least 5×, 10×, or 20× more recombinant oncolytic adenovirus compared to a similar method (e.g., an otherwise identical method) that comprises, in step (b), adherent culturing the infected A549 host cell in a serum-free medium, under conditions (e.g., in a replication permissive environment) to permit replication of the recombinant oncolytic adenovirus. In certain embodiments, the method results in at least 5×, 10×, or 20× more recombinant oncolytic adenovirus compared to a similar method (e.g., an otherwise identical method) that comprises, in step (b), suspension culturing the infected A549 host cell in a serum-containing medium, under conditions (e.g., in a replication permissive environment) to permit replication of the recombinant oncolytic adenovirus.

In another aspect, the invention provides a method for producing a recombinant oncolytic adenovirus comprising: (a) introducing a nucleic acid comprising a nucleotide sequence encoding a recombinant oncolytic adenovirus into an A549 host cell; and (b) suspension culturing the A549 host cell in a serum-free medium, under conditions (e.g., in a replication permissive environment) to permit production of the recombinant oncolytic adenovirus, thereby to produce the recombinant oncolytic adenovirus. In certain embodiments, the A549 host cell is a SF-BMAdR 281 A549 host cell. In certain embodiments, the A549 host cell is cultured for at least 3 days.

The method may further comprise, after step (b), the step of purifying the recombinant oncolytic adenovirus. The step of purifying the recombinant oncolytic adenovirus may comprise one or more of lysing the A549 host cell, nuclease treatment, and ion exchange chromatography, e.g., anion exchange chromatography. In certain embodiments, the step of purifying the recombinant oncolytic adenovirus comprises: (i) lysing the A549 host cell to produce a cell lysate; (ii) treating the cell lysate with nuclease to produce a treated cell lysate; and (iii) purifying the recombinant virus from the treated cell lysate by ion exchange chromatography, e.g., anion exchange chromatography.

The method may result in a greater yield of recombinant oncolytic adenovirus than a comparable method for producing a recombinant oncolytic adenovirus. For example, in certain embodiments, the method results in at least 5×, 10×, or 20× more recombinant oncolytic adenovirus compared to a similar method (e.g., an otherwise identical method) that comprises, in step (a), introducing a nucleic acid comprising a nucleotide sequence encoding a recombinant oncolytic adenovirus into a HEK293 host cell, and, in step (b), suspension culturing the HEK293 host cell in a serum-free medium, under conditions (e.g., in a replication permissive environment) to permit production of the recombinant oncolytic adenovirus. In certain embodiments, the method results in at least 5×, 10×, or 20× more recombinant oncolytic adenovirus compared to a similar method (e.g., an otherwise identical method) that comprises, in step (b), adherent culturing the A549 host cell in a serum-free medium, under conditions (e.g., in a replication permissive environment) to permit replication of the recombinant oncolytic adenovirus. In certain embodiments, the method results in at least 5×, 10×, or 20× more recombinant oncolytic adenovirus compared to a similar method (e.g., an otherwise identical method) that comprises, in step (b), suspension culturing the A549 host cell in a serum-containing medium, under conditions (e.g., in a replication permissive environment) to permit replication of the recombinant oncolytic adenovirus.

In certain embodiments, the recombinant oncolytic adenovirus comprises an E1a promoter having a deletion of a functional Pea3 binding site. For example, the virus may comprise a deletion of nucleotides corresponding to about-300 to about-250 upstream of the initiation site of E1a, e.g., a deletion of nucleotides corresponding to −305 to −255 or −304 to −255 upstream of the initiation site of E1a. In certain embodiments, the deletion comprises a deletion of nucleotides corresponding to 195-244 of the Ad5 genome (SEQ ID NO: 1), and/or the E1a promoter comprises the sequence GGTGTTTTGG (SEQ ID NO: 2).

In certain embodiments, the recombinant oncolytic adenovirus comprises an E1a promoter having a deletion of a functional TATA box, e.g., the deletion of an entire TATA box. For example, in certain embodiments, the virus comprises a deletion of nucleotides corresponding to −27 to −24, −31 to −24, −44 to +54, or −146 to +54 of the adenovirus type 5 E1a promoter, which correspond, respectively, to nucleotides 472 to 475, 468 to 475, 455 to 552, and 353 to 552 of the Ad5 genome (SEQ ID NO: 1). In certain embodiments, the virus comprises a polynucleotide deletion that results in a virus comprising the sequence CTAGGACTG (SEQ ID NO: 3), AGTGCCCG (SEQ ID NO: 8), or TATTCCCG (SEQ ID NO: 9), which result from joining the two polynucleotide sequences that would otherwise flank the deleted polynucleotide sequence.

In certain embodiments, the recombinant oncolytic adenovirus comprises a deletion of nucleotides corresponding to −29 to −26, −33 to −26, −44 to +52, or −148 to +52 of the adenovirus type 5 E1a promoter. In certain embodiments, the virus comprises a deletion of nucleotides corresponding to 353 to 552 of the Ad5 genome (SEQ ID NO: 1). In certain embodiments, the virus comprises a polynucleotide deletion that results in a virus comprising the sequence CTAGGACTG (SEQ ID NO: 3), which results from joining the two polynucleotide sequences that would otherwise flank the deleted polynucleotide sequence.

In certain embodiments, the recombinant oncolytic adenovirus comprises an E1a promoter having a deletion of a functional CAAT box, e.g., the deletion of an entire CAAT box. For example, in certain embodiments, the virus comprises a deletion of nucleotides corresponding to −76 to −68 of the adenovirus type 5 E1a promoter, which corresponds to nucleotides 423 to 431 of the Ad5 genome (SEQ ID NO: 1). In certain embodiments, the virus comprises a polynucleotide deletion that results in a virus comprising the sequence TTCCGTGGCG (SEQ ID NO: 10), which results from joining the two polynucleotide sequences that would otherwise flank the deleted polynucleotide sequence.

In certain embodiments, the recombinant oncolytic adenovirus comprises a nucleotide sequence encoding a transgene, which may, e.g., be inserted into an E1b-19K insertion site, wherein the E1b-19K insertion site is located between the start site of E1b-19K and the start site of E1b-55K. In certain embodiments, the E1b-19K insertion site is located between the start site of E1b-19K and the stop site of E1b-19K. In certain embodiments, the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent the start site of E1b-19K. In certain embodiments, the E1b-19K insertion site comprises a deletion of about 200 nucleotides, e.g., 202 or 203 nucleotides adjacent the start site of E1b-19K. In certain embodiments, the E1b-19K insertion site comprises a deletion corresponding to nucleotides 1714-1917 or 1714-1916 of the Ad5 genome (SEQ ID NO: 1). In certain embodiments, the nucleotide sequence encoding the transgene is inserted between nucleotides corresponding to 1714 and 1917 or between nucleotides corresponding to 1714 and 1916 of the Ad5 genome (SEQ ID NO: 1). In certain embodiments, the nucleotide sequence encoding the transgene is inserted between CTGACCTC (SEQ ID NO: 4) and TCACCAGG (SEQ ID NO: 5), e.g., the virus comprises, in a 5′ to 3′ orientation, CTGACCTC (SEQ ID NO: 4), the nucleotide sequence encoding the transgene, and TCACCAGG (SEQ ID NO: 5).

In certain embodiments, the nucleotide sequence encoding the transgene is not operably linked to an exogenous promoter sequence.

In certain embodiments, the transgene encodes a polypeptide selected from CD80, CD137L, IL-23, IL-23A/p19, p40, IL-27, IL-27A/p28, IL-27B/EBI3, ICAM-1, a TGF-ß trap, TGF-β, CD19, CD20, IL-1, IL-3, IL-4, IL-5, IL-6, IL-8, IL-9, CD154, CD86, BORIS/CTCFL, FGF, IL-24, MAGE, NY-ESO-1, acetylcholine, interferon-gamma, DKK1/Wnt, p53, thymidine kinase, an anti-PD-1 antibody heavy chain or light chain, and an anti-PD-L1 antibody heavy chain or light chain.

In certain embodiments, the recombinant virus, e.g., the recombinant oncolytic adenovirus, may selectively replicate in a hyperproliferative cell and/or selectively express the transgene in a hyperproliferative cell. The hyperproliferative cell may be a cancer cell.

In another aspect, the invention provides a recombinant virus, e.g., a recombinant oncolytic adenovirus, produced by a method disclosed herein.

In another aspect, the invention provides a method of treating cancer in a subject in need thereof, the method comprising administering to the subject an effective amount of a recombinant virus, e.g., a recombinant oncolytic adenovirus, produced by a method disclosed herein to treat the cancer in the subject.

These and other aspects and advantages of the invention are illustrated by the following figures, detailed description and claims.

### DESCRIPTION OF THE DRAWINGS

The invention can be more completely understood with reference to the following drawings.

FIG. 1 is a line graph depicting mean tumor volumes in mice following treatment with the indicated virus.

FIG. 2 is a line graph depicting progression free survival of mice treated with the indicated virus. Progression is defined as tumor volume exceeding 200 mm3.

FIG. 3 depicts viral production from a HEK-293 derived cell line and the SF-BMAdR 281 (A549 derived) cell line. No results were available for unmodified A549 cells because they could not be adapted to serum-free suspension culture.

### DETAILED DESCRIPTION

The invention is based, in part, upon the discovery that an A549 host cell, e.g., a SF-BMAdR 281 A549 host cell, can be used to produce large quantities of a recombinant virus, e.g., an oncolytic adenovirus. It has surprisingly has been found that certain recombinant viruses, e.g., recombinant oncolytic adenoviruses, grow to higher densities in a replication permissive environment in serum-free and suspension-adapted A549 cells than in HEK293 cells, which are widely used for viral vector production.

Accordingly, in one aspect, the invention provides a method for producing a recombinant virus comprising: (a) infecting an A549 host cell with a recombinant virus to produce an infected A549 host cell; and (b) suspension culturing the infected A549 host cell in a serum-free medium, under conditions (e.g., in a replication permissive environment) to permit replication of the recombinant virus, thereby to produce the recombinant virus. In certain embodiments, the recombinant virus is an adenovirus, e.g., a type 5 adenovirus, or an adeno-associated virus. In certain embodiments, the recombinant virus is a recombinant oncolytic virus. In certain embodiments, the recombinant virus is a recombinant oncolytic adenovirus.

In another aspect, the invention provides a method for producing a recombinant oncolytic adenovirus comprising: (a) infecting an A549 host cell with a recombinant oncolytic adenovirus to produce an infected A549 host cell, and (b) suspension culturing the infected A549 host cell in a serum-free medium, under conditions (e.g., in a replication permissive environment) to permit replication of the recombinant oncolytic adenovirus, thereby to produce the recombinant oncolytic adenovirus.

In another aspect, the invention provides a method for producing a recombinant oncolytic adenovirus comprising: (a) introducing a nucleic acid comprising a nucleotide sequence encoding a recombinant oncolytic adenovirus into an A549 host cell, and (b) suspension culturing the A549 host cell in a serum-free medium, under conditions (e.g., in a replication permissive environment) to permit production of the recombinant oncolytic adenovirus, thereby to produce the recombinant oncolytic adenovirus. The nucleic acid can be introduced into the cell using any method known in the art, e.g., liposome-based transfection, chemical-based transfection (e.g., utilizing calcium phosphate, cationic polymers, DEAE-5 dextran, or activated dendrimers), microinjection, electroporation, nanoparticles, or cell squeezing. The nucleic acid may, for example, be part of a plasmid, or may, for example, be part of more than one plasmid.

In certain embodiments of any of the foregoing methods, the A549 host cell is a SF-BMAdR 281 A549 host cell.

An A549 host cell, e.g., an infected A549 host cell, may be cultured for at least 1 day, at least 2 days, at least 3 days, at least 4 days, at least 5 days, at least 6 days, or at least 7 days.

Following production, viral particles are recovered from the culture and optionally purified. Typical purification steps may include centrifugation, e.g., cesium chloride gradient centrifugation, clarification, enzymatic treatment, e.g., nuclease or protease treatment, chromatographic steps, e.g., ion exchange chromatography, (e.g., anion exchange chromatography), or filtration steps. Accordingly, in certain embodiments, any of the foregoing methods further comprise, after step (b), the step of purifying a recombinant virus, e.g., a recombinant oncolytic adenovirus. The step of purifying the recombinant virus, e.g., the recombinant oncolytic adenovirus, may comprise lysing an A549 host cell, e.g., an infected A549 host cell, nuclease treatment, and/or ion exchange chromatography, e.g., anion exchange chromatography. In certain embodiments, the step of purifying the recombinant virus, e.g., the recombinant oncolytic adenovirus, comprises: (i) lysing an A549 host cell, e.g., an infected A549 host cell, to produce a cell lysate; (ii) treating the cell lysate with nuclease to produce a treated cell lysate; and (iii) purifying the recombinant virus from the treated cell lysate by ion exchange chromatography, e.g., anion exchange chromatography.

In certain embodiments, any of the foregoing methods may result in a greater yield of recombinant virus, e.g., recombinant oncolytic adenovirus, than a comparable method for producing a recombinant virus. For example, in certain embodiments, a method may result in greater yield of recombinant virus, e.g., recombinant oncolytic adenovirus, compared to a similar method that is the same method but for the use of a different host cell type. Viral yield can be assayed by any method known in the art, including, e.g., qPCR, immunocytochemistry, or a luciferase reporter assay.

For example, in certain embodiments, a method results in at least 2×, at least 3×, at least 4×, at least 5×, at least 10×, at least 15×, at least 20×, at least 25×, or at least 30× more recombinant virus, e.g., recombinant oncolytic adenovirus, compared to a similar method (e.g., an otherwise identical method) that comprises, in step (a), infecting a HEK293 host cell with a recombinant virus to produce an infected HEK293 host cell, and, in step (b), suspension culturing the infected HEK293 host cell in a serum-free medium, under conditions (e.g., in a replication permissive environment) to permit replication of the recombinant virus. In certain embodiments, a method results in at least 2×, at least 3×, at least 4×, at least 5×, at least 10×, at least 15×, at least 20×, at least 25×, or at least 30× more recombinant virus, e.g., recombinant oncolytic adenovirus, compared to a similar method (e.g., an otherwise identical method) that comprises, in step (a), introducing a nucleic acid comprising a nucleotide sequence encoding a recombinant oncolytic adenovirus into a HEK293 host cell, and, in step (b), suspension culturing the HEK293 host cell in a serum-free medium, under conditions (e.g., in a replication permissive environment) to permit replication of the recombinant virus.

In certain embodiments, the method may result in greater yield of recombinant virus, e.g., recombinant oncolytic adenovirus, compared to a similar method that is the same method but for the use of adherent culture in place of suspension culture. For example, in certain embodiments, the method results in at least 2×, at least 3×, at least 4×, at least 5×, at least 10×, at least 15×, at least 20×, at least 25×, or at least 30× more recombinant virus, e.g., recombinant oncolytic adenovirus, compared to a similar method (e.g., an otherwise identical method) that comprises, in step (b), adherent culturing an A549 host cell, e.g., an infected A549 host cell, in a serum-free medium, under conditions (e.g., in a replication permissive environment) to permit replication of the recombinant virus. In certain embodiments, the method may result in greater yield of recombinant virus, e.g., recombinant oncolytic adenovirus, compared to a similar method that is the same method but for the use of serum-containing media in place of serum-free media. For example, in certain embodiments, the method results in at least 2×, at least 3×, at least 4×, at least 5×, at least 10×, at least 15×, at least 20×, at least 25×, or at least 30× more recombinant virus e.g., recombinant oncolytic adenovirus, compared to a similar method (e.g., an otherwise identical method) that comprises, in step (b), suspension culturing an A549 host cell, e.g., an infected A549 host cell, in a serum-containing medium, under conditions (e.g., in a replication permissive environment) to permit replication of the recombinant virus.

In certain embodiments, a method further comprises contacting an A549 host cell with an epigenetic agent, e.g., a DNMT, HDAC, and/or tyrosine kinase inhibitor, Exemplary epigenetic agents include vorinostat, romidepsin, azacitidine, decitabine, RRx-001 and CUDC-101. In certain embodiments, a method further comprises contacting an A549 host cell with an interferon. In certain embodiments, a method further comprises contacting an A549 host cell with an antioxidant, e.g., vitamin C, vitamin E, glutathione, or N-acetylcysteine.

Various features and aspects of the invention are discussed in more detail below.

### I. Viruses

The term “virus” is used herein to refer any of the obligate intracellular parasites having no protein-synthesizing or energy-generating mechanism. The viral genome may be RNA or DNA. A recombinantly modified virus is referred to herein as a “recombinant virus.” A recombinant virus may, e.g., be modified by recombinant DNA techniques to be replication deficient, conditionally replicating, or replication competent, and/or be modified by recombinant DNA techniques to include expression of exogenous transgenes. Chimeric viral vectors which exploit advantageous elements of each of the parent vector properties (Sec, e.g., Feng et al. (1997) NATURE BIOTECHNOLOGY 15:866-870) may also be useful in the practice of the present invention. Although it is generally favored to employ a virus from the species to be treated, in some instances it may be advantageous to use vectors derived from different species that possess favorable pathogenic features.

In certain embodiments, the recombinant virus is an oncolytic virus, e.g., a virus that exhibits tumor-selective replication and/or viral mediated lysis. In certain embodiments, the oncolytic virus allows for selective expression of a gene, e.g., a transgene. For example, in certain embodiments, the virus permits expression of the gene in neoplastic cells, but attenuates expression in normal cells. In certain embodiments, the expression of the gene in a non-hyperproliferative cell is about 90%, about 80%, about 70%, about 60%, about 50%, about 40%, about 30%, about 20%, about 10%, or about 5% of the expression of in a hyperproliferative cell. In certain embodiments, the virus exhibits no detectable expression of the gene in a non-hyperproliferative cell. Gene expression may be determined by any appropriate method known in the art, e.g., Western blot or ELISA. The hyperproliferative cell may be a cancer cell, e.g., a carcinoma, sarcoma, leukemia, lymphoma, prostate cancer, lung cancer, gastrointestinal tract cancer, colorectal cancer, pancreatic cancer, breast cancer, ovarian cancer, cervical cancer, stomach cancer, thyroid cancer, mesothelioma, liver cancer, kidney cancer, skin cancer, head and neck cancer, or brain cancer cell.

In certain embodiments, the recombinant virus is an adenovirus or an adeno-associated virus. In certain embodiments, the recombinant virus is an adenovirus. Adenoviruses are medium-sized (90-100 nm), non-enveloped (naked), icosahedral viruses composed of a nucleocapsid and a double-stranded linear DNA genome. Adenoviruses replicate in the nucleus of mammalian cells using the host's replication machinery. The term “adenovirus” refers to any virus in the genus Adenoviridiae including, but not limited to, human, bovine, ovine, equine, canine, porcine, murine, and simian adenovirus subgenera. In particular, human adenoviruses includes the A-F subgenera as well as the individual serotypes thereof, the individual serotypes and A-F subgenera including but not limited to human adenovirus types 1, 2, 3, 4, 4a, 5, 6, 7, 8, 9, 10, 11 (Ad11a and Ad11p), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. Preferred are recombinant viruses derived from human adenovirus types 2 and 5. Unless stated otherwise, all adenovirus type 5 nucleotide numbers are relative to the NCBI reference sequence AC_000008.1, which is depicted herein in SEQ ID NO: 1.

The adenovirus replication cycle has two phases: an early phase, during which 4 transcription units E1, E2, E3, and E4 are expressed, and a late phase which occurs after the onset of viral DNA synthesis when late transcripts are expressed primarily from the major late promoter (MLP). The late messages encode most of the virus's structural protcins. The gene products of E1, E2 and E4 are responsible for transcriptional activation, cell transformation, viral DNA replication, as well as other viral functions, and are necessary for viral growth.

The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

In certain embodiments, the recombinant virus has one or more modifications to a regulatory sequence or promoter. A modification to a regulatory sequence or promoter comprises a deletion, substitution, or addition of one or more nucleotides compared to the wild-type sequence of the regulatory sequence or promoter.

In certain embodiments, the modification of a regulatory sequence or promoter comprises a modification of sequence of a transcription factor binding site to reduce affinity for the transcription factor, for example, by deleting a portion thereof, or by inserting a single point mutation into the binding site. In certain embodiments, the additional modified regulatory sequence enhances expression in neoplastic cells, but attenuates expression in normal cells.

In certain embodiments, the modified regulatory sequence is operably linked to a sequence encoding a protein. In certain embodiments, at least one of the adenoviral E1a and E1b genes (coding regions) is operably linked to a modified regulatory sequence. In certain embodiments, the E1a gene is operably linked to the modified regulatory sequence.

The E1a regulatory sequence contains five binding sites for the transcription factor Pea3, designated Pea3 I, Pea3 II, Pea3 III, Pea3 IV, and Pea3 V, where Pea3 I is the Pca3 binding site most proximal to the E1a start site, and Pea3 V is most distal. The E1a regulatory sequence also contains binding sites for the transcription factor E2F, hereby designated E2F I and E2F II, where E2F I is the E2F binding site most proximal to the E1a start site, and E2F II is more distal. From the E1a start site, the binding sites are arranged: Pea3 I, E2F I, Pea3 II, E2F II, Pea3 III, Pea3 IV, and Pea3 V.

In certain embodiments, at least one of these seven binding sites, or a functional binding site, is deleted. As used herein, a “functional binding site” refers to a binding site that is capable of binding to a respective binding partner, e.g., a transcription factor, e.g., a binding site that has at least 100%, at least 90%, at least 80%, at least 70%, at least 60%, at least 50%, or at least 40%, of the binding activity of a corresponding wild-type binding site sequence. As used herein, a “non-functional binding site” refers to a binding site that, e.g., has less than 30%, less than 20%, less than 10%, or 0% of the binding activity of a corresponding wild-type binding site sequence.

In certain embodiments, a recombinant adenovirus, e.g., a recombinant oncolytic adenovirus, comprises an E1a promoter having a deletion of a functional Pea3 binding site, e.g., the deletion of an entire Pea3 binding site. As used herein, a “functional Pea3 binding site” refers to a Pea3 binding site that is capable of binding to its respective transcription factor (e.g., Pea3), e.g., a Pea3 binding site that has at least 100%, at least 90%, at least 80%, at least 70%, at least 60%, at least 50%, or at least 40%, of the Pea3 binding activity of a corresponding wild-type Pea3 binding site sequence. As used herein, a “non-functional Pea3 binding site” refers to a Pea3 binding site that, e.g., has less than 30%, less than 20%, less than 10%, or 0% of the Pea3 binding activity of a corresponding wild-type Pea3 binding site sequence. Assays for determining whether a Pea3 binding site binds to Pea3 are known in the art. Exemplary binding assays include electrophoretic mobility shift assays, chromatin immunoprecipitation assays, and DNAse footprinting assays.

In certain embodiments, at least one Pea3 binding site, or a functional Pea3 binding site, is deleted. The deleted Pea3 binding site can be Pea3 I, Pea3 II, Pea3 III, Pea3 IV, and/or Pea3 V. In certain embodiments, the deleted Pea3 binding site is Pea3 II, Pea3 III, Pea3 IV, and/or Pea3 V. In certain embodiments, the deleted Pea3 binding site is Pea3 IV and/or Pea3 V. In certain embodiments, the deleted Pea3 binding site is Pea3 II and/or Pea3 III. In certain embodiments, the deleted Pea3 binding site is both Pea3 II and Pea3 III. In certain embodiments, the Pea3 I binding site, or a functional Pea3 I binding site, is retained.

In certain embodiments, at least one E2F binding site, or a functional E2F binding site, is deleted. In certain embodiments, at least one E2F binding site, or a functional E2F binding site, is retained. In certain embodiments, the retained E2F binding site is E2F I and/or E2F II. In certain embodiments, the retained E2F binding site is E2F II. In certain embodiments, the recombinant adenovirus, e.g., recombinant oncolytic adenovirus, may comprise a deletion of at least one E2F binding site, or a functional portion thereof, and not comprise a deletion of a Pea3 binding site. In certain embodiments, the total deletion consists essentially of one or more of Pea3 II, Pea3 III, Pea3 IV, and/or Pea3 V. In certain embodiments, the virus has a deletion of a 50 base pair region located from −304 to −255 upstream of the E1a initiation site, e.g., corresponding to 195-244 of the Ad5 genome (SEQ ID NO: 1), hereafter referred to as the TAV-255 deletion. In certain embodiments, the TAV-255 deletion results in an E1a promoter that comprises the sequence GGTGTTTTGG (SEQ ID NO: 2).

In certain embodiments, a recombinant adenovirus, e.g., a recombinant oncolytic adenovirus, comprises an E1a promoter having a deletion of a functional TATA box, e.g., the deletion of an entire TATA box. As used herein, a “functional TATA box” refers to a TATA box that is capable of binding to a TATA box binding protein (TBP), e.g., a TATA box that has at least 100%, at least 90%, at least 80%, at least 70%, at least 60%, at least 50%, or at least 40%, of the TBP binding activity of a corresponding wild-type TATA box sequence. As used herein, a “non-functional TATA box” refers to a TATA box that, e.g., has less than 30%, less than 20%, less than 10%, or 0% of the TBP binding activity of a corresponding wild-type TATA box sequence. Assays for determining whether a TBP binds to a TATA box are known in the art. Exemplary binding assays include electrophoretic mobility shift assays, chromatin immunoprecipitation assays, and DNAse footprinting assays.

For example, in certain embodiments, a recombinant adenovirus, e.g., a recombinant oncolytic adenovirus, comprises a deletion of nucleotides corresponding to −27 to −24, −31 to −24, −44 to +54, or −146 to +54 of the adenovirus type 5 E1a promoter, which correspond, respectively, to nucleotides 472 to 475, 468 to 475, 455 to 552, and 353 to 552 of the Ad5 genome (SEQ ID NO: 1). In certain embodiments, the virus comprises a deletion of nucleotides corresponding to −29 to −26, −33 to −26, −44 to +52, or −148 to +52 of the adenovirus type 5 E1a promoter. In certain embodiments, the virus comprises a deletion of nucleotides corresponding to 353 to 552 of the Ad5 genome (SEQ ID NO: 1). In certain embodiments, the virus comprises a polynucleotide deletion that results in a virus comprising the sequence CTAGGACTG (SEQ ID NO: 3), AGTGCCCG (SEQ ID NO: 8), or TATTCCCG (SEQ ID NO: 9), which result from joining the two polynucleotide sequences that would otherwise flank the deleted polynucleotide sequence. In certain embodiments, the virus comprises a polynucleotide deletion that results in a virus comprising the sequence CTAGGACTG (SEQ ID NO: 3),

In certain embodiments, a recombinant adenovirus, e.g., a recombinant oncolytic adenovirus, comprises an E1a promoter having a deletion of a functional CAAT box, e.g., the deletion of an entire CAAT box. As used herein, a “functional CAAT box” refers to a CAAT box that is capable of binding to a C/EBP or NF—Y protein, e.g., a CAAT box that has at least 100%, at least 90%, at least 80%, at least 70%, at least 60%, at least 50%, or at least 40%, of the a C/EBP or NF—Y binding activity of a corresponding wild-type CAAT box sequence. As used herein, a “non-functional CAAT box” refers to a CAAT box that, e.g., has less than 30%, less than 20%, less than 10%, or 0% of the a C/EBP or NF—Y binding activity of a corresponding wild-type CAAT box sequence. Assays for determining whether a C/EBP or NF—Y protein binds to a CAAT box are known in the art. Exemplary binding assays include electrophoretic mobility shift assays, chromatin immunoprecipitation assays, and DNAse footprinting assays.

For example, in certain embodiments, a recombinant adenovirus, e.g., a recombinant oncolytic adenovirus, comprises a deletion of nucleotides corresponding to −76 to −68 of the adenovirus type 5 E1a promoter, which correspond to nucleotides 423 to 431 of the Ad5 genome (SEQ ID NO: 1). In certain embodiments, the virus comprises a polynucleotide deletion that results in a virus comprising the sequence TTCCGTGGCG (SEQ ID NO: 10), which results from joining the two polynucleotide sequences that would otherwise flank the deleted polynucleotide sequence.

The adenoviral E1b-19k gene functions primarily as an anti-apoptotic gene and is a homolog of the cellular anti-apoptotic gene, BCL-2. Since host cell death prior to maturation of the progeny viral particles would restrict viral replication, E1b-19k is expressed as part of the E1 cassette to prevent premature cell death thereby allowing the infection to proceed and yield mature virions. Accordingly, in certain embodiments, a recombinant adenovirus, e.g., a recombinant oncolytic adenovirus, is provided that includes an E1b-19K insertion site, e.g., the recombinant adenovirus has a nucleotide sequence encoding a transgene inserted into an E1b-19K insertion site. In certain embodiments, the insertion site is located between the start site of E1b-19K (i.e., the nucleotide sequence encoding the start codon of E1b-19k, e.g., corresponding to nucleotides 1714-1716 of SEQ ID NO: 1) and the start site of E1b-55K (i.e., the nucleotide sequence encoding the start codon of E1b-55k, e.g., corresponding to nucleotides 2019-2021 of SEQ ID NO: 1). In certain embodiments, the E1b-19K insertion site is located between the start site of E1b-19K (i.e., the nucleotide sequence encoding the start codon of E1b-19k, e.g., corresponding to nucleotides 1714-1716 of SEQ ID NO: 1) and the stop site of E1b-19K (i.e., the nucleotide sequence encoding the stop codon of E1b-19k, e.g., corresponding to nucleotides 2242-2244 of SEQ ID NO: 1).

Throughout the description and claims, an insertion between two sites, for example, an insertion between (i) a start site of a first genc (e.g., E1b-19k) and a start site of a second gene, (e.g., E1b-55K), (ii) a start site of a first gene and a stop site of a second gene, (iii) a stop site of a first gene and start site of a second gene, or (iv) a stop site of first gene and a stop site of a second gene, is understood to mean that all or a portion of the nucleotides constituting a given start site or a stop site surrounding the insertion may be present or absent in the final virus. Similarly, an insertion between two nucleotides is understood to mean that the nucleotides surrounding the insertion may be present or absent in the final virus.

In certain embodiments, the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent the start site of E1b-19K. In certain embodiments, the E1b-19K insertion site comprises a deletion of about 200 nucleotides, e.g., 202 or 203 nucleotides adjacent the start site of E1b-19K. In certain embodiments, the E1b-19K insertion site comprises a deletion corresponding to nucleotides 1714-1917 or 1714-1916 of the Ad5 genome (SEQ ID NO: 1). In certain embodiments, a nucleotide sequence encoding a transgene is inserted between nucleotides corresponding to 1714 and 1917 or between nucleotides corresponding to 1714 and 1916 of the Ad5 genome (SEQ ID NO: 1). In certain embodiments, a nucleotide sequence encoding a transgene is inserted between CTGACCTC (SEQ ID NO: 4) and TCACCAGG (SEQ ID NO: 5), e.g., the recombinant adenovirus comprises, in a 5′ to 3′ orientation, CTGACCTC (SEQ ID NO: 4), a nucleotide sequence encoding a transgene, and TCACCAGG (SEQ ID NO: 5). CTGACCTC (SEQ ID NO: 4) and TCACCAGG (SEQ ID NO: 5) define unique boundary sequences for the E1b-19K insertion site within the Ad5 genome (SEQ ID NO: 1). Throughout the description and claims, a deletion adjacent a site, for example, a deletion adjacent a start site of a gene or a deletion adjacent a stop site of a gene, is understood to mean that the deletion may include a deletion of all, a portion, or none of the nucleotides constituting a given start site or a stop sitc.

In certain embodiments, a recombinant adenovirus, e.g., a recombinant oncolytic adenovirus, is provided that includes an E3 insertion site, e.g., the recombinant adenovirus has a nucleotide sequence encoding a transgene inserted into an E3 insertion site. In certain embodiments, the insertion site is located between the stop site of pVIII (i.e., the nucleotide sequence encoding the stop codon of pVIII, e.g., corresponding to nucleotides 27855-27857 of SEQ ID NO: 1) and the start site of Fiber (i.e., the nucleotide sequence encoding the start codon of Fiber, e.g., corresponding to nucleotides 31042-31044 of SEQ ID NO: 1). In certain embodiments, the E3 insertion site comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides. In certain embodiments, the E3 insertion site is located between the stop site of E3-10.5K (i.e., the nucleotide sequence encoding the stop codon of E3-10.5K, e.g., corresponding to nucleotides 29770-29772 of SEQ ID NO: 1) and the stop site of E3-14.7K (i.e., the nucleotide sequence encoding the stop codon of E3-14.7K, e.g., corresponding to nucleotides 30837-30839 of SEQ ID NO: 1). In certain embodiments, the E3 insertion site comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K. In certain embodiments, the E3 insertion site comprises a deletion of about 1050 nucleotides adjacent the stop site of E3-10.5K, e.g., the E3 insertion site comprises a deletion of 1063 or 1064 nucleotides adjacent the stop site of E3-10.5K. In certain embodiments, the E3 insertion site comprises a deletion corresponding to the Ad5 dl309 E3 deletion. In certain embodiments, the E3 insertion site comprises a deletion corresponding to nucleotides 29773-30836 of the Ad5 genome (SEQ ID NO: 1), or, a nucleotide sequence encoding a transgene is inserted between nucleotides corresponding to 29773 and 30836 of the Ad5 genome (SEQ ID NO: 1). In certain embodiments, a nucleotide sequence encoding a transgene is inserted between CAGTATGA (SEQ ID NO: 11) and TAATAAAAAA (SEQ ID NO: 12), e.g., the recombinant adenovirus comprises, in a 5′ to 3′ orientation, CAGTATGA (SEQ ID NO: 11), a nucleotide sequence encoding a transgene, and TAATAAAAAA (SEQ ID NO: 12). CAGTATGA (SEQ ID NO: 11) and TAATAAAAAA (SEQ ID NO: 12) define unique boundary sequences for an E3 insertion site within the Ad5 genome (SEQ ID NO: 1).

In certain embodiments, the E3 insertion site is located between stop site of E3-gp19K (i.e., the nucleotide sequence encoding the stop codon of E3-gp19K, e.g., corresponding to nucleotides 29215-29217 of SEQ ID NO: 1) and the stop site of E3-14.7K (i.e., the nucleotide sequence encoding the stop codon of E3-14.7K, e.g., corresponding to nucleotides 30837-30839 of SEQ ID NO: 1). In certain embodiments, the E3 insertion site comprises a deletion of from about 500 to about 1824, from about 500 to about 1500, from about 500 to about 1000, from about 1000 to about 1824, from about 1000 to about 1500, or from about 1500 to about 1824 nucleotides adjacent the stop site of E3-gp19K. In certain embodiments, the E3 insertion site comprises a deletion of about 1600 nucleotides adjacent the stop site of E3-gp19K. e.g., the E3 insertion site comprises a deletion of 1622 nucleotides adjacent the stop site of E3-gp19K. In certain embodiments, the E3 insertion site comprises a deletion corresponding to nucleotides 29218-30839 of the Ad5 genome (SEQ ID NO: 1). In certain embodiments, a nucleotide sequence encoding a transgene is inserted between nucleotides corresponding to 29218 and 30839 of the Ad5 genome (SEQ ID NO: 1). In certain embodiments, a nucleotide sequence encoding a transgene is inserted between TGCCTTAA (SEQ ID NO: 13) and TAAAAAAAAAT (SEQ ID NO: 14), e.g., the recombinant adenovirus comprises, in a 5′ to 3′ orientation, TGCCTTAA (SEQ ID NO: 13), a nucleotide sequence encoding a transgene, and TAAAAAAAAAT (SEQ ID NO: 14). TGCCTTAA (SEQ ID NO: 13) and TAAAAAAAAAT (SEQ ID NO: 14) define unique boundary sequences for an E3 insertion site within the Ad5 genome (SEQ ID NO: 1).

In certain embodiments, a recombinant adenovirus, e.g., a recombinant oncolytic adenovirus, comprises an E4 deletion. In certain embodiments, the E4 deletion is located between the start site of E4-ORF6/7 (i.e., the nucleotide sequence encoding the start codon of E4-ORF6/7, e.g., corresponding to nucleotides 34075-34077 of SEQ ID NO: 1) and the right inverted terminal repeat (ITR; e.g., corresponding to nucleotides 35836-35938 of SEQ ID NO: 1). In certain embodiments, the E4 deletion is located between the start site of E4-ORF6/7 and the start site of E4-ORF1 (i.e., the nucleotide sequence encoding the start codon of E4-ORF1, e.g., corresponding to nucleotides 35524-35526 of SEQ ID NO: 1). In certain embodiments, the E4 deletion comprises a deletion of a nucleotide sequence between the start site of E4-ORF6/7 and the start site of E4-ORF1. In certain embodiments, the E4 deletion comprises a deletion of from about 500 to about 2500, from about 500 to about 2000, from about 500 to about 1500, from about 500 to about 1000, from about 1000 to about 2500, from about 1000 to about 2000, from about 1000 to about 1500, from about 1500 to about 2500, from about 1500 to about 2000, or from about 2000 to about 2500 nucleotides. In certain embodiments, the E4 deletion comprises a deletion of from about 250 to about 1500, from about 250 to about 1250, from about 250 to about 1000, from about 250 to about 750, from about 250 to about 500, from 500 to about 1500, from about 500 to about 1250, from about 500 to about 1000, from about 500 to about 750, from 750 to about 1500, from about 750 to about 1250, from about 750 to about 1000, from about 1000 to about 1500, or from about 1000 to about 1250 nucleotides adjacent the start site of E4-ORF6/7. In certain embodiments, the E4 deletion comprises a deletion of about 1450 nucleotides adjacent the start site of E4-ORF6/7, e.g., the E4 deletion comprises a deletion of about 1449 nucleotides adjacent the start site of E4-ORF6/7. In certain embodiments, the E4 deletion comprises a deletion corresponding to nucleotides 34078-35526 of the Ad5 genome (SEQ ID NO: 1).

Nucleic acids encoding viral genes can be incorporated into plasmids and introduced into host cells through conventional transfection or transformation techniques. Specific production and purification conditions will vary depending upon the virus and the production system employed. For adenovirus, the traditional method for the generation of viral particles is co-transfection followed by subsequent in vivo recombination of a shuttle plasmid (usually containing a small subset of the adenoviral genome and optionally containing a potential transgene an expression cassette) and an adenoviral helper plasmid (containing most of the entire adenoviral genome). Alternative technologies for the generation of adenovirus include utilization of the bacterial artificial chromosome (BAC) system, in vivo bacterial recombination in a recA=bacterial strain utilizing two plasmids containing complementary adenoviral sequences, and the yeast artificial chromosome (YAC) system.

### II. Therapeutic Transgenes

A recombinant virus, e.g., a recombinant oncolytic adenovirus, produced using a method disclosed herein may comprise an exogenous nucleotide sequence that encodes for a therapeutic transgene. The term “transgene” refers to an exogenous gene or polynucleotide sequence. The term “therapeutic transgene” refers to a transgene, which when replicated and/or expressed in or by the virus imparts a therapeutic effect in a target cell, body fluid, tissue, organ, physiological system, or subject.

The therapeutic transgene may encode a therapeutic nucleic acid, e.g., an antisense RNA or ribozyme RNA. The therapeutic transgene may encode a therapeutic peptide or polypeptide, e.g., an apoptotic agent, antibody, CTL responsive peptide, cytokine, cytolytic agent, cytotoxic agent, enzyme, heterologous antigen expressed on the surface of a tumor cell to elicit an immune response, immunostimulatory or immunomodulatory agent, interferon, lytic peptide, oncoprotein, polypeptide which catalyzes processes leading to cell death, polypeptide which complements genetic defects in somatic cells, tumor suppressor protein, vaccine antigen, or any combination thereof.

In certain embodiments, the therapeutic transgene encodes a therapeutic polypeptide selected from CD80, CD137L, IL-23, IL-23A/p19, p40, IL-27, IL-27A/p28, IL-27B/EBI3, ICAM-1, a TGF-β trap, TGF-β, CD19, CD20, IL-1, IL-3, IL-4, IL-5, IL-6, IL-8, IL-9, CD154, CD86, BORIS/CTCFL, FGF, IL-24, MAGE, NY-ESO-1, acetylcholine, interferon-gamma, DKK1/Wnt, p53, thymidine kinase, an anti-PD-1 antibody heavy chain or light chain, and an anti-PD-L1 antibody heavy chain or light chain.

### III. Pharmaceutical Compositions

For therapeutic use, a recombinant virus, e.g., a recombinant oncolytic adenovirus, produced using a method disclosed herein is preferably combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing recombinant viruses can be presented in a dosage unit form and can be prepared by any suitable method. A pharmaceutical composition should be formulated to be compatible with its intended route of administration. Examples of routes of administration are intravenous (IV), intraarterial, intradermal, inhalation, transdermal, topical, transmucosal, and rectal administration. A preferred route of administration is IV infusion. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The term “effective amount” as used herein refers to the amount of an active component (e.g., the amount of a recombinant virus) sufficient to effect beneficial or desired results. An effective amount can be administered in one or more administrations, applications or dosages and is not intended to be limited to a particular formulation or administration route.

In certain embodiments, a therapeutically effective amount of active component is in the range of 0.1 mg/kg to 100 mg/kg, e.g., 1 mg/kg to 100 mg/kg, 1 mg/kg to 10 mg/kg. In certain embodiments, a therapeutically effective amount of the recombinant virus is in the range of 10² to 10¹⁵ plaque forming units (pfus), e.g., 10² to 10¹⁰, 10² to 10⁵, 10⁵ to 10¹⁵, 10⁵ to 10¹⁰, or 10¹⁰ to 10¹⁵ plaque forming units. The amount administered will depend on variables such as the type and extent of disease or indication to be treated, the overall health of the patient, the in vivo potency of the virus, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the virus, and the disease being treated. Exemplary dosing frequencies are once per day, once per week and once every two weeks. A preferred route of administration is parenteral, e.g., intravenous infusion.

### IV. Therapeutic Uses

A recombinant virus, e.g., a recombinant oncolytic adenovirus produced using a method disclosed herein, can be used to treat various medical indications, for example, cancers. As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma).

In certain embodiments, the cancer is selected from melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, prostate cancer, gastroesophageal cancer, colorectal cancer, testicular cancer, bladder cancer, ovarian cancer, hepatocellular carcinoma, cholangiocarcinoma, brain cancer, endometrial cancer, neuroendocrine cancer, and pancreatic cancer.

In certain embodiments, the cancer is selected from nasopharyngeal cancer, basal cell carcinoma, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, neuroendocrine, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

In certain embodiments, a recombinant virus, e.g., a recombinant oncolytic adenovirus, is administered to the subject in combination with one or more therapies, e.g., surgery, radiation, chemotherapy, immunotherapy, hormone therapy, or virotherapy. In certain embodiments, a recombinant virus is administered in combination with a tyrosine kinase inhibitor, e.g., erlotinib. In certain embodiments, a recombinant virus of the invention is administered in combination with a checkpoint inhibitor, e.g., an anti-CTLA-4 antibody, an anti-PD-1 antibody, or an anti-PD-L1 antibody. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies include, for example, atezolizumab (Tecentriq®, Genentech), duvalumab (AstraZeneca), MEDI4736, avelumab, and BMS 936559 (Bristol Myers Squibb Co.).

The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Throughout the description, where viruses, compositions and systems are described as having, including, or comprising specific components, or where processes and methods are described as having, including, or comprising specific steps, it is contemplated that, additionally, there are compositions, devices, and systems of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a virus, a composition, a system, a method, or a process described herein can be combined in a variety of ways without departing from the spirit and scope of the present invention, whether explicit or implicit herein. For example, where reference is made to a particular virus, that virus can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

At various places in the present specification, viruses, compositions, systems, processes and methods, or features thereof, are disclosed in groups or in ranges. It is specifically intended that the description include each and every individual subcombination of the members of such groups and ranges. By way of other examples, an integer in the range of 1 to 20 is specifically intended to individually disclose 1, 2, 3, 4, 5, 6, 7, 8, 9, 10, 11, 12, 13, 14, 15, 16, 17, 18, 19, and 20.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1: Production of an Oncolytic Adenovirus

This Example describes the production of a recombinant oncolytic adenovirus in A549 cells.

An adenovirus type 5 virus was constructed that carries the deletion of a nucleotide region located from −304 to −255 upstream of the E1a initiation site, which renders E1a expression cancer-selective (as previously described in U.S. Pat. No. 9,073,980). The resulting virus is hereafter referred to as TAV.

TAV was further modified to carry an approximately 200 base pair deletion in the E1b-19k region. The resulting virus is hereafter referred to as TAV-Δ19k. The nucleotide sequence of the modified E1b-19k region is as follows, with residual bases from fused Sall and Xhol sites underlined:

(SEQ ID NO: 6)
ATCTTGGTTACATCTGACCTCGTCGAGTCACCAGGCGCTTTTCCAA

TAV-Δ19k was modified to include a nucleotide sequence encoding a mouse TGF-β trap (a fusion protein of the mouse TGFβ type II receptor and mouse IgG1) in the modified E1b-19k region. The resulting virus is hereafter referred to as TAV-mTGFβ-Trap. The nucleotide sequence encoding the TGF-β trap is as follows:

(SEQ ID NO: 7)
ATGGGTCGGGGGCTGCTCCGGGGCCTGTGGCCGCTGCATATCGTCCTGTG
GACGCGCATCGCCAGCACGATCCCGCCGCACGTTCCCAAGTCGGTTAACA
GTGATGTCATGGCCAGCGACAATGGCGGTGCGGTCAAGCTTCCACAGCTG
TGCAAGTTTTGCGATGTGAGACTGTCCACTTGCGACAACCAGAAGTCCTG
CATGAGCAACTGCAGCATCACGGCCATCTGTGAGAAGCCGCATGAAGTCT
GCGTGGCCGTGTGGAGGAAGAACGACAAGAACATTACTCTGGAGACGGTT
TGCCACGACCCCAAGCTCACCTACCACGGCTTCACTCTGGAAGATGCCGC
TTCTCCCAAGTGTGTCATGAAGGAAAAGAAAAGGGCGGGCGAGACTTTCT
TCATGTGTGCCTGTAACATGGAAGAGTGCAACGATTACATCATCTTTTCG
GAAGAATACACCACCAGCAGTCCCGACAGCACCAAGGTGGACAAGAAAAT
TGTGCCCAGGGATTGTGGTTGTAAGCCTTGCATATGTACAGTCCCAGAAG
TATCATCTGTCTTCATCTTCCCCCCAAAGCCCAAGGATGTGCTCACCATT
ACTCTGACTCCTAAGGTCACGTGTGTTGTGGTAGACATCAGCAAGGATGA
TCCCGAGGTCCAGTTCAGCTGGTTTGTAGATGATGTGGAGGTGCACACAG
CTCAGACGCAACCCCGGGAGGAGCAGTTCAACAGCACTTTCCGCTCAGTC
AGTGAACTTCCCATCATGCACCAGGACTGGCTCAATGGCAAGGAGTTCAA
ATGCAGGGTCAACAGTGCAGCTTTCCCTGCCCCCATCGAGAAAACCATCT
CCAAAACCAAAGGCAGACCGAAGGCTCCGCAGGTGTACACCATTCCACCT
CCCAAGGAGCAGATGGCCAAGGATAAAGTCAGTCTGACCTGCATGATAAC
AGACTTCTTCCCTGAAGACATTACTGTGGAGTGGCAGTGGAATGGGCAGC
CAGCGGAGAACTACAAGAACACTCAGCCCATCATGGACACAGATGGCTCT
TACTTCGTCTACAGCAAGCTCAATGTGCAGAAGAGCAACTGGGAGGCAGG
AAATACTTTCACCTGCTCTGTGTTACATGAGGGCCTGCACAACCACCATA
CTGAGAAGAGCCTCTCCCACTCTCCTGGTAAATGA

SF-BMAdR 281 A549 cells (purchased from National Research Council of Canada) were cultured in serum-free media (Hyclone SFM4Transfx-293) in suspension culture in shake flasks. After growth to a density of 2×10⁶ cells/mL in a total volume of 100 mL, the cells were centrifuged and resuspended in 100 mL of fresh SFM4Transfx-293 media. 50 mL of the resuspended culture was infected with the TAV-Δ19k adenovirus, and 50 mL of the resuspended culture was infected with the TAV-mTGFβ-Trap adenovirus. The cells were maintained in suspension culture in shake flasks for three days to allow for viral replication, and the cultures were then lysed with freeze-thaw cycles to produce cell lysate.

The viruses were then purified from the cell lysate by centrifugation, nuclease treatment, anion exchange chromatography, and dialysis into a buffer appropriate for in vivo administration (10 mM Tris, 1 mM MgCl₂, 3% sucrose, pH 8).

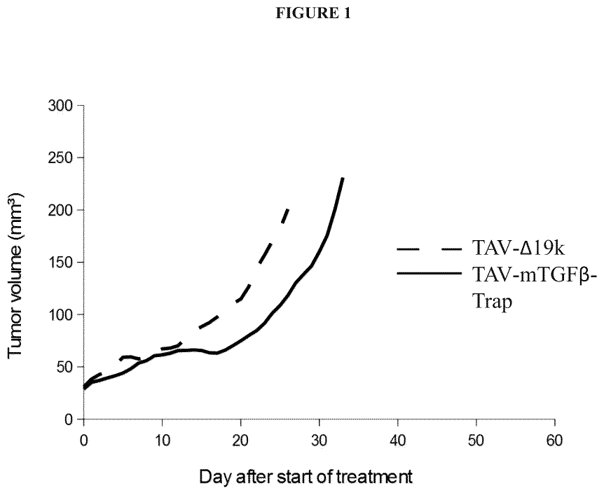

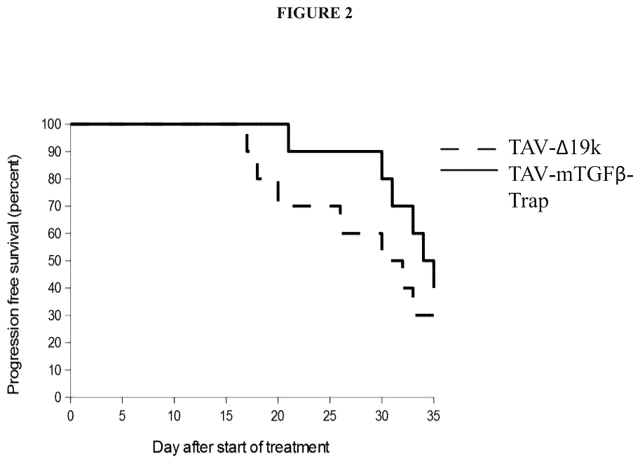

The viruses were then tested for efficacy in vivo. Adult 129S4 mice were injected subcutaneously with 1×10⁶ ADS-12 cells, a pulmonary cancer cell line, and allowed to form subcutaneous tumors. After the tumors grew large enough to treat, 10 mice each were treated with intratumoral injections of either the TAV-Δ19k adenovirus or the TAV-mTGFβ-Trap adenovirus. Three doses of 1×10⁹ IU of each virus were administered every four days. Mean tumor volume in mice treated with each virus is depicted in FIG. 1, and progression free survival of mice treated with each virus is depicted in FIG. 2.

### Example 2: Production of an Oncolytic Adenovirus

This Example describes the production of a recombinant oncolytic adenovirus in A549 derived cells relative to HEK-293 derived cells.

An adenovirus type 5 virus was constructed that carries the deletion of a nucleotide region located from −304 to −255 upstream of the E1a initiation site, which renders E1a expression cancer-selective (as previously described in U.S. Pat. No. 9,073,980). The resulting virus is hereafter referred to as TAV.

TAV was further modified to carry an approximately 200 base pair deletion in the E1b-19k region. The resulting virus is hereafter referred to as TAV-Δ19k. The nucleotide sequence of the modified E1b-19k region is as follows, with residual bases from fused Sall and Xhol sites underlined:

(SEQ ID NO: 6)
ATCTTGGTTACATCTGACCTCGTCGAGTCACCAGGCGCTTTTCCAA

TAV-Δ19k was modified to include a nucleotide sequence encoding a human TGF-β trap (a fusion protein of the human TGFß type II receptor and human IgG1) in the modified E1b-19k region. The resulting virus is hereafter referred to as TAV-hTGFβ-Trap.

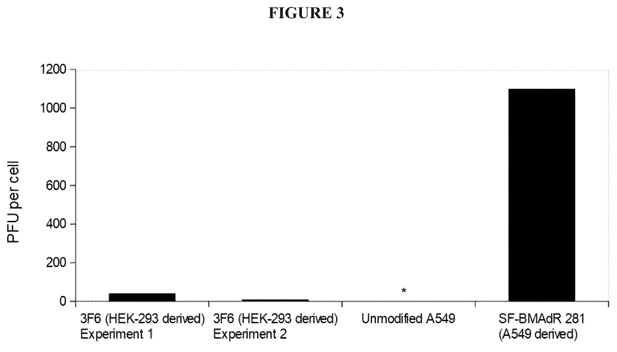

TAV-hTGFβ-Trap adenovirus was produced in both HEK-293 cells (293-3F6) and A549 cells (SF-BMAdR). HEK-293 cells cultured in serum-free medium (SFM4Transfx-293) at 5×10⁵ cells/mL were infected with TAV-hTGFβ-Trap at a multiplicity of infection (MOI) of 3. At 4 days post-infection the yield was 42 PFU/cell. In a separate experiment, HEK-293 cells cultured in serum-free medium (SFM4Transfx-293) at 1×10⁶ cells/mL were infected with TAV-hTGFβ-Trap at an MOI of 3. At 4 days post-infection the yield was less than 10 PFU/cell. A549 cells cultured in serum-free medium (SFM4Transfx-293) at 1×10⁶ cells/mL were infected with TAV-hTGFβ-Trap at an MOI of 3. At 4 days post-infection the yield was 1100 PFU/cell. Unmodified A549 cells could not be adapted to grow in the same serum-free medium (SFM4Transfx-293) in suspension culture. Viral production from these cell lines is depicted in FIG. 3.

Together, these results show that A549 derived host cells, e.g., SF-BMAdR A549 host cells, produce greater yields of certain oncolytic viruses, e.g., the TAV-hTGFβ-Trap adenovirus.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

What is claimed is:

1. A composition comprising an A549 host cell adapted to suspension culturing in a serum-free medium infected with a recombinant oncolytic adenovirus,
wherein the recombinant oncolytic adenovirus comprises an E1a gene comprising an E1a protein coding region operably linked to a modified regulatory sequence comprising (i) deletion of a functional Pea3 binding site, (ii) deletion of a functional E2F binding site, (iii) deletion of a functional TATA box, (iv) deletion of a functional CAAT box, (v) deletion of the nucleotides corresponding to the nucleotides from 195-244 of the Ad5 genome (SEQ ID NO: 1), or (vi) has two, three, or four of (i), (ii), (iii), and (iv).

2. The composition of claim 1, wherein the recombinant oncolytic adenovirus is a type 5 adenovirus (Ad5).

3. The composition of claim 1, wherein the A549 host cell is a SF-BMAdR 281 A549 cell.

4. The composition of claim 1, wherein the A549 host cell is cultured for at least 3 days.

5. The composition of claim 1, further comprising, after step (b), the step of purifying the recombinant oncolytic adenovirus.

6. The composition of claim 5, wherein the step of purifying the recombinant oncolytic adenovirus comprises lysing the A549 host cell.

7. The composition of claim 5, wherein the step of purifying the recombinant oncolytic adenovirus comprises nuclease treatment.

8. The composition of claim 5, wherein the step of purifying the recombinant oncolytic adenovirus comprises ion exchange chromatography.

9. The composition of claim 8, wherein the step of purifying the recombinant oncolytic adenovirus comprises: (i) lysing the infected A549 host cell to produce a cell lysate; (ii) treating the cell lysate with nuclease to produce a treated cell lysate; and (iii) purifying the recombinant virus from the treated cell lysate by ion exchange chromatography.

10. The composition of claim 1, wherein the E1a promoter has the deletion of a functional Pea3 binding site.

11. The composition of claim 1, wherein the E1a promoter has the deletion of a functional TATA box.

12. The composition of claim 1, wherein the recombinant oncolytic adenovirus comprises a polynucleotide deletion that results in a virus sequence comprising the junction sequence corresponding to CTAGGACTG (SEQ ID NO: 3), AGTGCCCG (SEQ ID NO: 8) and/or TATTCCCG (SEQ ID NO: 9) of the Ad5 genome (SEQ ID NO: 1).

13. The composition of claim 1, wherein the E1a promoter has the deletion of a functional CAAT box.

14. The composition of claim 1, wherein the recombinant oncolytic adenovirus comprises a nucleotide sequence encoding a transgene.

15. The composition of claim 14, wherein the nucleotide sequence is inserted into an E1b-19K insertion site, and wherein the E1b-19K insertion site is located between the start site of E1b-19K and the stop site of E1b-19K.

16. The composition of claim 14, wherein the transgene is not operably linked to an exogenous promoter sequence and/or the recombinant virus selectively expresses the transgene in a hyperproliferative cell.

17. The composition of claim 14, wherein the transgene encodes a polypeptide selected from CD80, CD137L, IL-23, IL-23A/p19, p40, IL-27, IL-27A/p28, IL-27B/EBI3, ICAM-1, a TGF-β Trap, TGF-β, CD19, CD20, IL-1, IL-3, IL-4, IL-5, IL-6, IL-8, IL-9, CD154, CD86, BORIS/CTCFL, FGF, IL-24, MAGE, NY-ESO-1, acetylcholine, interferon-gamma, DKK1/Wnt, p53, thymidine kinase, an anti-PD-1 antibody heavy chain or light chain, and an anti-PD-L1 antibody heavy chain or light chain.

18. The composition of claim 1, wherein the Pea3 binding site is selected from Pea3 I, Pea3 II, Pea3 III, Pea3 IV, and Pea3 V.

19. The composition of claim 1, wherein the E2F binding site is selected from E2F I and E2F II.
